(19) United States
(12) Patent Application Publication
(10) Pub. No.: US 2011/0268660 A1
(43) Pub. Date: Nov. 3, 2011

(54) METHOD FOR DETECTING DYSPLASIA

(76) Inventors: Antonios Danikas, Amersham (GB); Benedicte Guilbert, Amersham (GB)

(21) Appl. No.: 13/058,345

(22) PCT Filed: Aug. 14, 2009
(86) PCT No.: PCT/EP2009/060571

§ 371 Date: Feb. 10, 2011

Related U.S. Application Data
(60) Provisional application No. 61/089,073, filed on Aug. 15, 2008.

(30) Foreign Application Priority Data
Aug. 15, 2008 (GB) 0814960.1

(51) Int. Cl. A61K 49/00

(52) U.S. Cl. 424/9.6; 424/9.1

(57) ABSTRACT

The present invention provides a method of imaging useful in the determination of sites of dysplasia in patients suffering from Barrett's oesophagus. The method comprises the use of an optical imaging agent comprising a vector which targets the extracellular domain of EGFR, the vector also being selective for EGFR over Her2. The vector is labelled with an optical reporter suitable for in vivo imaging using light in the green to near-infrared wavelength 500-1200 nm. Also provided are novel optical imaging agents suitable for use in the method.

### FIELD OF THE INVENTION

The present invention provides a method of imaging useful in the determination of sites of dysplasia in patients suffering from Barrett's oesophagus. The method comprises the use of an optical imaging agent comprising a vector which targets the extracellular domain of EGFR, the vector also being selective for EGFR over Her2. The vector is labelled with an optical reporter suitable for in vivo imaging using light in the green to near-infrared wavelength 500-1200 nm. Also provided are novel optical imaging agents suitable for use in the method.

### BACKGROUND TO THE INVENTION

Oesophageal cancer represents less than 5% of all reported cancer cases, but ca. 30,000 new such cases are diagnosed per annum in the USA and the survival rate is low (see below). Oesophageal cancer can be divided into two major types, squamous cell carcinoma and adenocarcinoma, depending on the type of cells that are malignant. Barrett's oesophagus is a pre-malignant condition which is associated with an increased risk of development of oesophageal cancer; especially adenocarcinoma [Kiesslich et al, Clin. Gastroenterol. Hepatol., 4, 979-987 (2006)]. Chronic reflux increases risk for Barrett's oesophagus, and it has therefore been suggested that gastro oesophageal reflux (GERD) is a risk factor for oesophageal cancer.

Adenocarcinoma of the oesophagus is more prevalent than squamous cell carcinoma in the USA and Western Europe. Oesophageal cancer can be a treatable disease but is rarely curable. The overall 5-year survival rate is between 5% and 30%. Early diagnosis of oesophageal cancer improves the survival rate of the patient. Primary treatment includes surgery alone or chemotherapy in combination with radiation. Chemotherapy used in treatment of oesophageal cancer includes 5-fluorouracil and cisplatin. Lack of precise pre-operative staging is a major clinical problem.

The presence of low grade dysplasia (i.e. abnormal tissue growth) in Barrett's oesophagus is a risk factor for the development of oesophageal cancer, but surveillance currently relies on histopathology [Lim et al, Endoscopy, 39, 581-7 (2007)]. Diagnosis of dysplasia in Barrett's oesophagus is currently via random four-quadrant biopsies every 1 to 2 cm (the Seattle protocol), which is time-consuming and costly [DaCosta et al, Best Pract. Res. Clin. Gastroenterol., 20(1), 41-57 (2006)]. Dysplasia in Barrett's oesophagus is not normally visible during routine endoscopy [Endlicher et al, Gut, 48, 314-319 (2001)].

U.S. Pat. No. 6,035,229 (Washington Research Foundation) describes a system for detecting Barrett's oesophagus utilizing an illumination and imaging probe at the end of a catheter. The document does not disclose an optical contrast agent.

Staining of Barrett's oesophagus tissue in vivo has been compared with staining of biopsy samples in vitro, using the dye methylene blue in the detection of highly dysplastic or malignant tissue [Canto et al, Endoscopy, 33, 391-400 (2001)].

Kiesslich et al [Clin. Gastroenterol. Hepatol., 4, 979-987 (2006)] reported on the use of fluorescein to aid the detection of Barrett's epithelium and associated neoplasia using confocal laser endomicroscopy.

WO 2005/058371 discloses optical imaging contrast agents for imaging of oesophageal cancer and Barrett's oesophagus in vivo. The contrast agents have an affinity for a biological target which is abnormally expressed in Barrett's oesophagus. The contrast agents of WO 2005/058371 are preferably of formula:

V-L-R
where:
V is one or more vector moieties having affinity for an abnormally expressed target in oesophageal cancer or Barrett's oesophagus;
L is a linker moiety or a bond; and
R is one or more reporter moieties detectable in optical imaging.

A wide range of targets is described, which are either overexpressed or downregulated in either Barrett's oesophagus, squamous cell carcinoma or adenocarcinoma. The target is preferably selected from: E-cadherin, CD44, P62/c-myc (HGF receptor), p53 and EGFR/erB-2 (claim 5). The vector (V) is stated to be preferably selected from peptides, peptoid moieties, oligonucleotides, oligosaccharides, fat-related compounds and traditional organic drug-like small molecules. The reporter (R) is preferably a dye that interacts with light in the wavelength region from the ultraviolet to the near-infrared part of the electromagnetic spectrum.

The epidermal growth factor (EGF) and EGR receptor (EGFR) are a ligand-receptor pair. The human epidermal growth factor receptor (HER) family consists of 4 closely-related ErbB receptor tyrosine kinases:
EGFR (also known as ErbB-1 or HER-1);
ErbB-2 (also known as HER-2 or Neu);
ErbB-3 (also known as HER-3); and
ErbB-4 (also known as HER-4).

The EGFR is a 170 kDa protein located at the cell surface, consisting of an amino-terminal extracellular ligand-binding domain, a single hydrophobic transmembrane helix, and an intracellular domain which contains the tyrosine kinase domain and a carboxy-terminal region containing critical tyrosine residues and receptor regulatory motifs. Binding of ligands such as EGF to the extracellular domain results in receptor dimerisation, activation and receptor autophosphorylation in several C-terminal tyrosine residues. These phosphorylated tyrosines serve as binding sites for a number of cytoplasmic signal-transducing molecules. Activation of these pathways downstream of the EGFR leads to cell proliferation, differentiation, migration/motility, adhesion, protection from apoptosis, enhanced survival, and gene transcription. EGFR is known to be abnormally activated in many epithelial tumours [Mendolsohn et al, Semin. Oncol., 33(4), 369-385 (2006)].

Cai et al [Eur. J. Nucl. Med. Mol. Imaging, 35, 186-208 (2008)] have reviewed approaches to multimodality imaging of human EGF receptor (EGFR, HER2, HER3 and HER4), based on PET, SPECT, optical and MRI.

Barrett's Oesophagus is a condition characterised by replacement of squamous oesophageal epithelium with columnar epithelium (metaplasia). A proportion of Barrett's Oesophagus patients will develop oesophageal adenocarcinoma, and the critical step in this process is the formation of dysplasia. Therefore patients diagnosed with dysplasia undergo therapy which can include anti-reflux medication, endoscopic mucosal ablation and surgical resection, depending on the severity of the disease (FIG. 1; summarised from “Guidelines for the Diagnosis and Management of Barrett's Columnar-lined Oesophagus”, UK Society of Gastroenterology, August 2005; www.bsg.org.uk):

Treated patients are followed up at 6monthly intervals to assess response to therapy. Responders (i.e. patients where the evidence is that the therapy is being effective), are treated as low-risk patients, while continued/additional treatment is considered for non-responders.

Curvers et al [Gastroenterol., 134, 670-679 (2008)] compared various diagnostic imaging techniques in Barrett's Oesophagus: magnifying white light endoscopy (WLE); narrow-band imaging (NBI); indigo carmine chromoendoscopy (ICC) and acetic acid chromoendoscopy (AAC). They concluded that, whilst some techniques, gave improved image quality, they did not improve the observer's ability to distinguish areas of dysplasia from those without dysplasia. There is therefore a need for more reliable earlier diagnosis of the subset of Barrett's Oesophagus patients at risk of developing oesophageal adenocarcinoma, especially identifying sites of dysplasia from normal tissue.

### THE PRESENT INVENTION

The present invention provides a method of imaging useful in the determination of sites of dysplasia in patients suffering from Barrett's oesophagus. The method comprises the use of an optical imaging agent comprising a vector which targets the extracellular domain of EGFR. The vector is labelled with an optical reporter suitable for in vivo imaging using light in the green to near-infrared wavelength 500-1200 nm.

The present invention shows that EGFR expression shows a significant upregulation at sites of dysplasia in Barrett's oesophagus patients. The degree of expression was found to correlate with the degree of dysplasia. Consequently, the imaging method of the invention can be used to guide biopsy (i.e. obtaining tissue samples from the sites of dysplasia to establish in vitro whether the abnormality is benign or cancerous). It can also be used to assist patient diagnosis and stratification, and to guide and/or monitor therapy (eg. tissue ablation). The present invention facilitates the diagnosis of “at risk” patients at an early stage of disease, and thus permits early clinical intervention to initiate therapy where appropriate.

Also provided are novel optical imaging agents suitable for use in the method. Targeting extracellular as opposed to intracellular EGFR has the advantage that the imaging agent does not need to cross the cell membrane, so that access to the desired target is not governed by transport processes across the cell membrane and the permeability of the imaging agent. Optical imaging is preferred for two reasons:
(i) it is compatible with current diagnostic procedures;
(ii) it minimises patient exposure to radiation.

### DETAILED DESCRIPTION OF THE INVENTION

In a first aspect, the present invention provides a method of in vivo imaging, for use in the determination of sites of potential dysplasia in a patient suffering from Barrett's oesophagus, said method comprising:
(i) provision of an imaging agent which comprises a vector which targets the extracellular domain of EGFR, said vector being labelled with an optical reporter imaging moiety suitable for imaging the mammalian body in vivo using light of wavelength 500-1200 nm;
(ii) carrying out optical imaging at least a portion of the oesophagus of said patient with the imaging agent from step (i);
(iii) making a determination from the imaging of step (ii) whether there is increased uptake of the imaging agent relative to background at one or more locations of the patient's oesophagus;
(iv) when the determination of step (iii) shows increased uptake for at least one such location, that location is identified as a site of potential dysplasia.

By the term “determination” is meant the identification of the location and extent of sites of increased uptake relative to oesophageal tissue background of the imaging agent.

By the term “patient” is meant a living mammalian, preferably human subject.

By the term “suffering from Barrett's Oesophagus” is meant that the patient has already been diagnosed, or is suspected to be suffering from Barrett's Oesophagus. The diagnosis would have been made on the basis of clinical symptoms plus typically confirmation using first-line endoscopy. Such first line endoscopy is currently carried out with white light, in order to collect random, four quadrant biopsies. Histological assessment of these biopsies confirms the degree of disease.

By the term “imaging agent” is meant a compound suitable for imaging the human body in vivo. The imaging may be invasive (eg. intra-operative or endoscopic) or non-invasive. A preferred imaging method is endoscopy, since that permits irradiation of the surface of the oesophagus with light, and detection of any fluorescence without intervening tissues to attenuate the light emitted by the tissue, and hence also without the need for surgical intervention to facilitate imaging. Thus, the imaging is preferably carried out on the intact body of the patient in vivo with minimal intervention.

The term “dysplasia” has its conventional meaning, i.e. abnormal tissue growth. Such growth can be benign, pre-cancerous or cancerous. The method of the present invention is preferably used at the pre-cancerous stage, i.e. for patients who have not been previously diagnosed with oesophageal adenocarcinoma. The phrase ‘potential dysplasia’ refers to a suspected abnormality detected via the imaging alone—actual dysplasia would typically be confirmed by tissue sampling (eg. by biopsy) and detailed testing of the tissue sample(s) in vitro. The imaging agents and methods of the present invention are thus aids to diagnosis.

The term “vector” has its conventional meaning in the field of in vivo diagnostic imaging, i.e. a compound which targets a particular biological site, such as a receptor or enzyme.

By the term “targets the extracellular domain of EGFR” is meant that the vector has a substantially higher affinity for EGFR compared to background tissue, and other potential targets or binding partners. The vector is able to bind to the target with high affinity (with a Ki value in the range 0 to 50 nM, preferably 1 to 20 nM, most preferably less than 10 nM). The vector is also specific for the extracellular domain of EGFR, and consequently has little or no affinity for the transmembrane or intracellular domains of EGFR. The EGFR is preferably human EGFR.

By the term “labelled with” is meant means that a functional group of the vector is conjugated to the optical reporter. Preferably the conjugation is via a covalent chemical bond.

By the term “optical reporter imaging moiety” is meant a fluorescent dye or chromophore which is capable of detection either directly or indirectly in an optical imaging procedure using light of green to near-infrared wavelength (500-1200 nm, preferably 600-1000 nm). Preferably, the reporter has fluorescent properties and is more preferably a fluorescent dye. Since the optical reporter must be suitable for imaging the mammalian body in vivo, it must also be biocompatible. By the term “biocompatible ” is meant non-toxic and hence suitable for administration to the mammalian body, especially the human body without adverse reaction, or pain or discomfort on administration.

The optical reporter is any moiety capable of detection either directly or indirectly in an optical imaging procedure. The reporter might be a light scatterer (eg. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

The optical reporter may optionally be conjugated to the vector via a linker group (L), where L is a synthetic linker group of formula -(A)ₘ-:
wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
and each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;

Optical imaging modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

The imaging agent of step (i) can be delivered by conventional routes of patient administration, such as intravenous or oral, as is known in the art. Methods of formulating drugs for more efficient delivery to the oesophagus are known in the art [Batchelor, Pharmaceut. Res., 22(2), 175-181 (2005] and Collaud et al, J. Control. Rel., 123, 203-210 (2007)], and hence the imaging agents of step (i) of the present invention may optionally be delivered via such formulations.

The vector may comprise either an endogenous or exogenous ligand for the extracellular region of EGFR, and is preferably synthetic.

Preferred Aspects.

The molecular weight of the imaging agent is preferably up to 20,000 Daltons (20 kDa), more preferably up to 15 kDa, most preferably up to 12 kDa, with up to 6 kDa being the ideal. Larger molecules such as antibodies have significantly higher molecular weight (ca. 150 kDa for an intact antibody; ca. 60 kDa for an FAb fragment; and ca. 30 kDa for an scFv fragment). The pharmacokinetics of such species is believed to be less suitable for imaging, due to slow clearance and poor tissue penetration.

The vector is preferably selective for EGFR over Her2. The term “selective for EGFR over Her2” means that the binding affinity of the vector to EGFR compared to Her2 is greater by a factor which allows selective imaging of EGFR during endoscopy.

Vectors which comprise an endogenous ligand for the extracellular region of EGFR are preferably chosen from
(i) human EGF (hEGF) or fragment thereof;
(ii) TGF-alpha or fragment thereof;
(iii) amphiregulin.

EGF is a 6 kDa protein that binds the EGFR extracellular domain with a K_{D}˜1 nM. Recombinant human EGF is commercially available from the Sigma Chemical Company. EGF has several amine sites suitable for conjugation with the optical reporter. Thus, when labelling EGF, the labelling site is not specifically targeted and can occur at various positions. hEGF contains 2 lysine, 5 glutamic acid and 7 aspartic acid residues. Therefore the optical reporter can be conjugated to the amino group of 2 lysine residues or at the N-terminus. EGF can be labelled with an optical reporter as described by Ke et al for the cyanine dye Cy5.5 [Cancer Res., 63, 7870-7875 (2003)], or Adams et al for the cyanine dye Cy5.5 and IRDye® 800CW-NHS [J. Biomed. Opt., 12(2) 024017 (2007)]. EGF has been described as having strong mitogenic and neoangiogenic activity, hence is a less preferred vector of the invention. 10 to 15-mer fragments of EGF which bind to the extracellular region of EGFR such as EGF₂₁₋₃₁ [Lutsenko, New Res. Biotechnol. Med., 211-220 (2006)] are also within the scope of the present invention.

TGF-α is a 50 amino acid peptide (molecular weight 5.5 kDa) that binds with high affinity (K_{D}˜1 nM) to EGFR. 10 to 15-mer fragments of TGF-α which bind to the extracellular region of EGFR, such as TGF₂₂₋₃₁ are also within the scope of the present invention. Recombinant human TGF-α is commercially available from the Sigma Chemical Company. TGF-α is preferably labelled with the optical reporter at either the N-terminus, or at the amino group of the 4-Lys residue.

Amphiregulin (AR; also known as keratinocyte autocrine factor) is an endogenous ligand selective for EGFR (HER1). Native AR proteins contain either 78 or 84 amino acid residues with both N- and O-linked oligosaccharides. A C-terminal extended form of AR, having a leucine-86 residue instead of methionine, named AR₁₋₉₀(leu86) (molecular weight ca. 9.4 kDa) has an improved binding affinity for EGFR compared to natural AR₁₋₈₄. [Adam et al., Biochim. Biophys. Acta, 1266, 83-90 (1995)].

A 98 amino acid residue (long form) of AR is commercially available from R&D Systems. A preferred AR of the present invention is AR₁₋₉₀(leu86).

Vectors which comprise an exogenous ligand for the extracellular region of EGFR are preferably chosen from

(i) 9-20-mer peptides containing the peptide sequence CKSPEPQHC (GE9);

(ii) 12-20-mer peptides containing the peptide sequence LHLWVPEPWTQT (GE10);

(iii) 12-20-mer peptides containing the peptide sequence YHWYGYTPQNVI (GE11);

(iv) 17-20-mer peptides containing the peptide sequence MLYNPTTYQMDVNPEGK (Inherbin 1);

(v) 17-20-mer peptides containing the peptide sequence LVYNKLTFQLEPNPHTK (Inherbin 3);

(vi) an Affibody™;

(vii) a Nanobody™;

(viii) 6-15-mer peptides containing the peptide sequence LARLLT (D4).

Peptides of the present invention, can be obtained by conventional solid phase peptide synthesis as described in P. Lloyd-Williams, F Albericio and E. Girald; Chemical Approaches to the Synthesis of Peptides and Proteins, CRC Press, 1997. GE9 is preferably cyclised (via the 2 cysteine residues), since the cyclic form is expected to exhibit increased stability compared to the open chain form. For GE9, the optical reporter can be attached to either the C-terminus or N-terminus via a linker group. The peptide GE11 is described by Li et al [FASEB J., 19, 1978-1985 (2005)]. For GE11, a suitable site for conjugation of the optical reporter is the amine group of the N-terminus of the peptide (as described by Li et al for fluorescein isothiocyanate). It is also possible to conjugate the optical reporter to the C-terminus of GE11 or to a linker attached to the C-terminus or N-terminus of GE11. It is, however, also possible to include an additional lysine or cysteine residue of the 12-20 mer peptide, or a linker group, and conjugate the optical reporter to the aminobutyl side chain of the lysine residue, or the thiol side chain of the cysteine residue. Preferred sites for labelling the peptides GE9 or GE10 are the N-terminus or a lysine residue as described for GE11.

The peptides Inherbin 1 and Inherbin 3 are described in WO 2007/115571 (Enkam Pharmaceuticals A/S). Preferred sites for labelling Inherbin 1 and Inherbin 3 with the optical reporter are the N-terminus, a lysine residue or cysteine residue as described for GE11.

The remaining amino acid residues of the peptides of exogenous ligands (i)-(v) may be made up of any amino acid. By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

By the term “peptide” is meant a compound comprising two or more amino acids, as defined above, linked by a peptide bond (ie. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids.

Affibody™ molecules are based on the 58 amino acid residue domain derived from one of the IgG-binding domains of staphylococcal protein A. Affibodies may be used in monomer or dimer form, and have been reviewed by Nygren [FEBS J., 275, 2668-2676 (2008)] and Nilsson et al [Curr. Opin. Drug. Disc. Dev., 10, 167-175 (2007)]. Friedman et al have reported an Affibody dimer molecule (Z_{EGFR:955})₂ with specific binding to the extracellular domain of EGFR [Protein Eng. Des. Select., 20(4), 189-199 (2007)]. Friedman et al also describe three selected monomer affibodies, Z_{EGFR:942}, Z_{EGFR:948} and Z_{EGFR:955} which bind selectively to the extracellular domain of EGFR. Further studies on dye-labelled and radioiodinated (Z_{EGFR:955})₂ were reported by Nordberg et al [Nucl. Med. Biol., 34, 609-618 (2007)]. Nordberg et al concluded that the affibody molecule (Z_{EGFR:955})₂ should be considered for EGFR-based radionuclide imaging. A second generation affibody monomer Z_{EGFR:1907} with improved EGFR binding affinity (improved about 20 fold; K_{D} 5.3 nM by Biacore analysis) has been reported by Orlova et al [Cancer Biother. Biopharm., 22(5), 573-584 (2007); J. Lab. Comp. Radiopharm., 50(Suppl.1): S70 (2007)].

The relatively small size of these Affibodies should allow better target tissue penetration and blood clearance compared to antibodies which are 10 to 20 times larger (˜150 kDa). Affibodies also have the advantage that they are stable under a range of pH conditions (pH 5.5 to 11). The affibodies are preferably labelled with the optical reporter via conjugation at the C-terminal cysteine residue with a maleimide-functionalised reporter [as described by Friedman et al above for (Z_{EGFR:955})₂], or via a covalent amide bond to the reporter. Preferred affibodies of the present invention are Z_{EGFR:942}, Z_{EGFR:948}, Z_{EGFR:955} and Z_{EGFR:1907} in monomer or dimer form, especially Z_{EGFR:1907}.

Camelids produce functional antibodies devoid of light chains in which the single N-terminal domain is fully capable of antigen binding. These single-domain antibody fragments (VHHs or nanobodies) have several advantages for biotechnological applications. Nanobodies have been reviewed by Muyldermans [J. Biotechnol., 74, 277-302 (2001)]. Their production and applications have been reviewed more recently by Harmsen et al [App. Microbiol. Biotechnol., 77, 13-22 (2007)]. Nanobodies specific for extracellular EGFR have been described by Gainkam et al [J. Nucl. Med., 49, 788-795 (2008)]; Huang et al [Mol. Imaging Biol., 10, 167-175 (2008)] and Roovers et al [Cancer Immunol. Immunother., 56, 303-317 (2007)]. Gainkam et al concluded that ^{99m}Tc-labelled 7C12 was a promising tumour imaging agent. Preferred nanobodies for use in the present invention are those named 7C12, 7D12 and 8B6.

D4 peptides are described by Song [FASEB J., 23(5), 1396-1404 (2009) and WO 2009/059450].

More preferred vectors of the invention are chosen from: TGF-alpha, Affibodies specific for EGFR, and the peptides GE9, GE10, GE11, D4, Inherbin 1 and Inherbin 3. Most preferred vectors of the invention are chosen from: Affibodies, the peptides GE9, GE10 or GE11 and Inherbin 1 or Inherbin 3.

Preferred organic chromophoric and fluorophoric reporters include groups having an extensive delocalized electron system, eg. cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots). Preferably, the optical reporter of the present invention does not comprise a metal complex, and is preferably a synthetic organic dye.

Particular examples of chromophores which may be used include: fluorescein, sulforhodamine 101 (Texas Red), rhodamine B, rhodamine 6G, rhodamine 19, indocyanine green, the cyanine dyes Cy2, Cy3, Cy3.5, Cy5, Cy5.5, Cy7, Marina Blue, Pacific Blue, Oregon Green 88, Oregon Green 514, tetramethylrhodamine, and Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

The imaging moiety is preferably an optical reporter, more preferably a fluorescent green to near-infrared dye. Such NIR dyes suitably have their absorption maximum in the green to near-infrared wavelength 500-1200 nm. The NIR dye is preferably a cyanine dye or benzopyrylium dyes. Preferred cyanine dyes which are fluorophores are of Formula I:

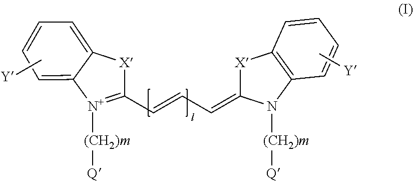

wherein:
each X′ is independently selected from: —C(CH₃)₂, —S—, —O— or —C[(CH₂)ₐCH₃][(CH₂)_{b}M]-, wherein a is an integer of value 0 to 5, b is an integer of value 1 to 5, and M is group G or is selected from SO₃M¹ or H;
each Y′ independently represents 1 to 4 groups selected from the group consisting of: H, —CH₂NH₂, —SO₃M¹, —CH₂COOM¹, —NCS, F and a group G, and wherein the Y′ groups are placed in any of the positions of the aromatic ring;
Q′ is independently selected from the group consisting of: H, SO₃M¹, NH₂, COOM¹, ammonium, ester groups, benzyl and a group G;
M¹ is H or B^{c}; where B^{c} is a biocompatible cation;
l is an integer from 1 to 3;
and m is an integer from 1 to 5;
wherein at least one of X′, Y′ and Q′ comprises a group G;
G is a reactive or functional group suitable for attaching to the vector.

By the term “biocompatible cation” (B^{c}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

The G group reacts with a complementary group of the vector forming a covalent linkage between the cyanine dye fluorophore and the vector. G may be a reactive group that may react with a complementary functional group of the peptide, or alternatively may include a functional group that may react with a reactive group of the vector. Examples of reactive and functional groups include: active esters; isothiocyanate; maleimide; haloacetamide; acid halide; hydrazide; vinylsulphone; dichlorotriazine; phosphoramidite; hydroxyl; amino; sulphydryl; carbonyl; carboxylic acid and thiophosphate. Preferably G is an active ester.

By the term “activated ester” or “active ester” is meant an ester derivative of the associated carboxylic acid which is designed to be a better leaving group, and hence permit more facile reaction with nucleophile, such as amines. Examples of suitable active esters are: N-hydroxysuccinimide (NHS), sulpho-succinimidyl ester, pentafluorophenol, pentafluorothiophenol, para-nitrophenol, hydroxybenzotriazole and PyBOP (ie. benzotriazol-1-yl-oxytripyrrolidinophosphonium hexafluorophosphate). Preferred active esters are N-hydroxysuccinimide or pentafluorophenol esters, especially N-hydroxysuccinimide esters.

Cyanine dyes which are more preferred are of Formula Ia:

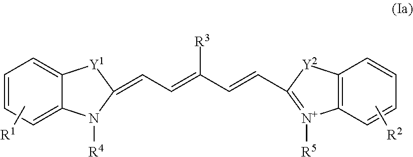

where:
   Y¹ and Y² are independently —O—, —S—, —NR⁶— or —CR⁷R⁸— and are chosen such that at least one of Y¹ and Y² is —CR⁷R⁸—; R¹ and R² are independently H, —SO₃M¹ or R^{a};
   R³ is H, C₁₋₅ alkyl, C₁₋₆ carboxyalkyl or an R^{a} group;
   R⁴ to R⁶ are independently C₁₋₅ alkyl, C₁₋₆ carboxyalkyl or R^{a};
   R⁷ is H or C₁₋₃ alkyl;
   R⁸ is R^{a} or C₁₋₆ carboxyalkyl;
   R^{a} is C₁₋₄ sulfoalkyl;
   where M¹ is as defined in Formula I;
with the proviso that the cyanine dye of Formula Ia comprises at least one R^{a} group and a total of 1 to 6 sulfonic acid substituents from the R¹, R² and R^{a} groups.

By the term “sulfonic acid substituent” is meant a substituent of formula —SO₃M¹, where M¹ is as defined above. Preferred dyes of Formula Ia have 3 to 6 sulfonic acid substituents. The —SO₃M¹ substituent is covalently bonded to a carbon atom, and the carbon atom may be aryl (such as the R¹ or R² groups), or alkyl (ie. an R^{a} group). In Formula Ia, the R^{a} groups are preferably of formula —(CH₂)ₖSO₃M¹, where M¹ is as defined above, and k is an integer of value 1 to 4. k is preferably 3 or 4.

Particularly preferred cyanine dyes are of Formula Ib:

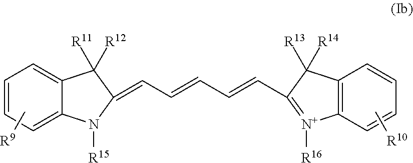

where:
   R⁹ and R¹⁰ are independently H or SO₃M¹, and at least one of R⁹ and R¹⁰ is SO₃M¹;
   R¹¹ and R¹² are independently C₁₋₄ alkyl or C₁₋₆ carboxyalkyl;
   R¹³, R¹⁴, R¹⁵ and R¹⁶ are independently R^{b} groups;
   wherein R^{b} is C₁₋₄ alkyl, C₁₋₆ carboxyalkyl or —(CH₂)_{q}SO₃M¹, where q is an integer of value 3 or 4;
   where M¹ is as defined for Formulae I and Ia;
   with the proviso that the cyanine dye has a total of 1 to 4 SO₃M¹ substituents in the R⁹, R¹⁰ and R^{b} groups.

Preferred cyanine dyes of Formula Ib are chosen such that at least one C₁₋₆ carboxyalkyl group is present, in order to facilitate conjugation to the vector.

Preferred individual cyanine dyes of Formula Ib are summarised in Table 1:

TABLE 1
chemical structures of individual cyanine dyes.

Dye name

| | Cy5(1) | Cy5(2) | Cy5** | Alexa647 |
|---|---|---|---|---|
| R⁹ | H | SO₃H | SO₃H | SO₃H |
| R¹⁰ | SO₃H | SO₃H | SO₃H | SO₃H |
| R¹¹ | CH₃ | CH₃ | CH₃ | R^{f} |
| R¹² | CH₃ | CH₃ | CH₃ | CH₃ |
| R¹³ | CH₃ | CH₃ | CH₃ | CH₃ |
| R¹⁴ | CH₃ | CH₃ | —(CH₂)₄SO₃H | CH₃ |
| R¹⁵ | R^{f} | R^{f} | R^{f} | —(CH₂)₃SO₃H |
| R¹⁶ | CH₃ | Et | —(CH₂)₄SO₃H | —(CH₂)₃SO₃H |

where R^{f} = —(CH₂)₅COOH.

Especially preferred cyanine dyes are of Formula Ic:

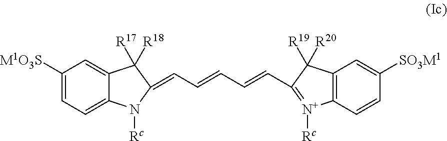

where:
R^{c} is independently an R^{a} group or C₁₋₆ carboxyalkyl;
R¹⁷ to R²⁰ are independently C₁₋₅ alkyl or an R^{c} group, and are chosen such that either R¹⁷═R¹⁸═R^{c} or R¹⁹═R²⁰═R^{d}, where R^{d} is C₁₋₂ alkyl;
R^{a} and M¹ are as defined above for Formula I.

Especially preferred cyanine dyes of Formulae Ib and Ic are Cy5** and Alexa647, with Cy5** being the ideal.

The term “benzopyrylium dye” has its conventional meaning. Suitable benzopyrylium dyes of the present invention are denoted Bzp^{M} and are of Formula II:

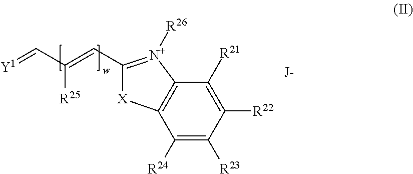

where:
   Y¹ is a group of Formula Y^{a} or Y^{b}

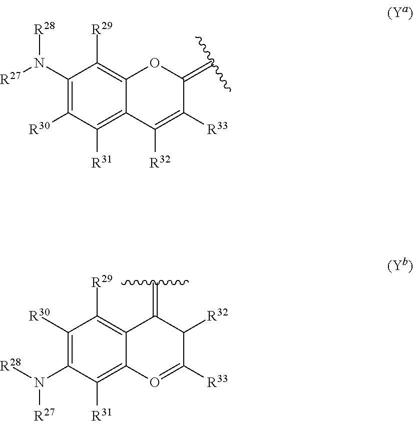

R²¹-R²⁴ and R²⁹-R³³ are independently selected from H, —SO₃M¹, Hal, R^{g} or C₃₋₁₂ aryl;
R²⁵ is H, C₁₋₄ alkyl, C₁₋₆ carboxyalkyl, C₃₋₁₂ arylsulfonyl, Cl, or R²⁵ together with one of R²⁶, R³⁴, R³⁵ or R³⁶ may optionally form a 5- or 6-membered unsaturated aliphatic, unsaturated heteroaliphatic or aromatic ring;
R²⁶ and R³⁶ are independently R^{g} groups;
R²⁷ and R²⁸ are independently C₁₋₄ alkyl, C₁₋₄ sulfoalkyl or C₁₋₆ hydroxyalkyl or optionally together with one or both of R²⁹ and/or R³⁰ may form a 5- or 6-membered N-containing heterocyclic or heteroaryl ring;
X is —CR³⁴R³⁵—, —O—, —S—, —Se—, —NR³⁶— or —CH═CH—, where R³⁴ to R³⁶ are independently R^{g} groups;
R^{g} is C₁₋₄ alkyl, C₁₋₄ sulfoalkyl, C₁₋₆ carboxyalkyl or C₁₋₆ hydroxyalkyl;
w is 1 or 2;
J is a biocompatible anion;
where M¹ is as defined for Formula I;
with the proviso that Bzp^{M} comprises at least one sulfonic acid substituent chosen from the R²¹ to R³⁶ groups.

By the term “biocompatible anion” (J) is meant a negatively charged counterion which forms a salt with an ionised, positively charged group (in this case an indolinium group), where said negatively charged counterion is also non-toxic and hence suitable for administration to the mammalian body, especially the human body. The counterion (J⁻) represents an anion which is present in a molar equivalent amount, thus balancing the positive charge on the Bzp^{M} dye. The anion (J) is suitably singly- or multiply-charged, as long as a charge-balancing amount is present. The anion is suitably derived from an inorganic or organic acid. Examples of suitable anions include: halide ions such as chloride or bromide; sulphate; nitrate; citrate; acetate; phosphate and borate. A preferred anion is chloride.

The benzopyrylium dye (Bzp^{M}) of Formula II is a fluorescent dye or chromophore which is capable of detection either directly or indirectly in an optical imaging procedure using light of green to near-infrared wavelength (500-1200 nm, preferably 550-1000 nm, more preferably 600-800 nm). Preferably, the Bzp^{M} has fluorescent properties.

Suitable imaging agents of the invention are those wherein the Bzp^{M} is of Formula IIa or IIb:

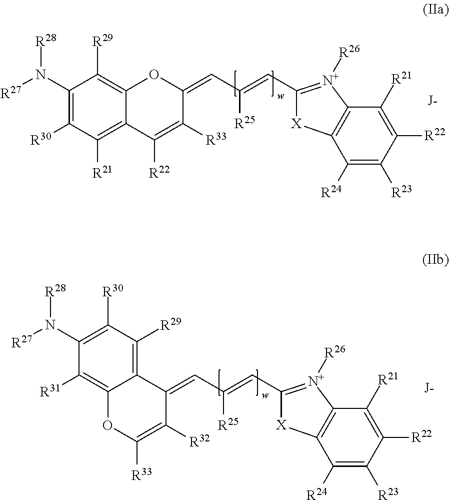

where X, w, J and R²¹-R³³ are as defined for Formula II.

When R²⁵ together with one of R²⁶/R³⁴-R³⁶ forms a 5- or 6-membered unsaturated aliphatic, unsaturated heteroaliphatic or aromatic ring, suitable such aromatic rings include: phenyl, furan, thiazole, pyridyl, pyrrole or pyrazole rings. Suitable unsaturated rings comprise at least the C═C to which R²⁵ is attached.

When R²⁷ and/or R²⁸ together with one or both of R²⁹ and/or R³⁰ form a 5- or 6-membered N-containing heterocyclic or heteroaryl ring, suitable such rings include: thiazole, pyridyl, pyrrole or pyrazole rings or partially hydrogenated versions thereof. preferably pyridyl or dihydropyridyl.

### Preferred Features of the benzopyrylium Dye

The vector is preferably attached at positions R²⁵, R²⁶, R³⁴, R³⁵ or R³⁶ of the Bzp^{M} of Formula II, more preferably at R²⁶, R³⁴, R³⁵ or R³⁶ most preferably at R²⁶, R³⁴ or R³⁵. In order to facilitate the attachment the relevant R²⁵, R²⁶, R³⁴, R³⁵ or R³⁶ substituent is preferably C₁₋₆ carboxyalkyl, more preferably C₃₋₆ carboxyalkyl.

The benzopyrylium dye (Bzp^{M}) preferably has at least 2 sulfonic acid substituents, more preferably 2 to 6 sulfonic acid substituents, most preferably 2 to 4 sulfonic acid substituents. Preferably, at least one of the sulfonic acid substituents is a C₁₋₄ sulfoalkyl group. Such sulfoalkyl groups are preferably located at positions R²⁶, R²⁷, R²⁸, R³⁴, R³⁵ or R³⁶; more preferably at R²⁶, R²⁷, R²⁸, R³⁴ or R³⁵; most preferably at R²⁶ together with one or both of R²⁷ and R²⁸ of Formula II. The sulfoalkyl groups of Formula II, are preferably of formula —(CH₂)ₖSO₃M¹, where M¹ is H or B^{c}, k is an integer of value 1 to 4, and B^{c} is a biocompatible cation (as defined above). k is preferably 3 or 4.

In Formula II, w is preferably 1. R²⁵ is preferably H or C₁₋₄ carboxyalkyl, and is most preferably H. X is preferably —CR³⁴R³⁵— or —NR³⁶—, and is most preferably —CR³⁴R³⁵—.

Preferred Bzp^{M} dyes are of Formula III:

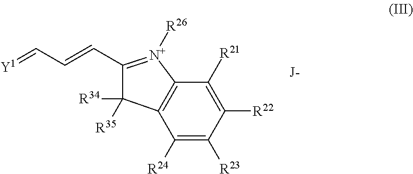

where Y¹, R²¹-R²⁴, R²⁶, R³⁴, R³⁵ and J are as defined for Formula II.

Suitable dyes of Formula III are of Formula IIIa or IIIb:

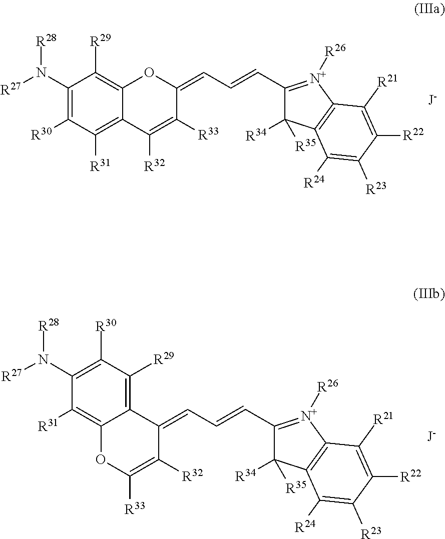

Preferred R²¹-R²⁴ and R²⁶-R³³ groups of Formulae III, IIIa and IIIb are as described above for formulae IIa and IIb. In Formulae III, IIIa and IIIb, R³⁴ and R³⁵ are preferably chosen such that one is an R^{j} group and the other is an R^{k} group. R^{j} is C₁₋₂ alkyl, most preferably methyl. R^{k} is C₁₋₄ alkyl, C₁₋₆ carboxyalkyl or C₁₋₄ sulfoalkyl, preferably C₃₋₆ carboxyalkyl or —(CH₂)ₖSO₃M¹ where k is chosen to be 3 or 4.

Preferably the dyes of Formula III have a C₁₋₆ carboxyalkyl substituent to permit facile covalent attachment to the vector.

In Formula II or III, when R²⁷ and/or R²⁸ together with one or both of R²⁹ and/or R³⁰ form a 5- or 6-membered N-containing heterocyclic or heteroaryl ring, preferred such rings are pyridyl or dihydropyridyl. A preferred such Y¹ group wherein an R²⁸ group has been cyclised with R³⁰ is of Formula Y^{c}:

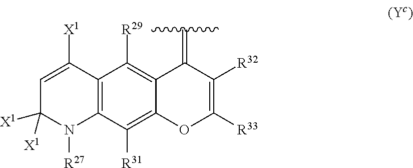

A preferred such Y¹ group wherein both R²⁷ and R²⁸ group have been cyclised is of Formula Y^{d}:

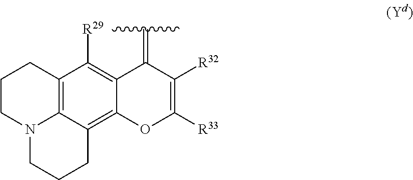

where:
R²⁷, R²⁹ and R³¹-R³³ are as defined above;
each X¹ is independently H or C₁₋₄ alkyl.

In Formula Y^{c}, it is preferred that:

each X¹ is CH₃;

R²⁹═R³¹═H;

R³² is H;

R³³ is CH₃ or —C(CH₃)₃, more preferably —C(CH₃)₃.

In Formula Y^{d}, it is preferred that:

R²⁹═H;

R³² is H;

R³³ is preferably CH₃ or —C(CH₃)₃, more preferably —C(CH₃)₃.

It is preferred that the —NR²⁷R²⁸ group of Formula II is either:
(i) in open chain form, ie. the R²⁷/R²⁸ groups are not cyclised with one or both of R²⁹/R³⁰ Preferred such R²⁷ and R²⁸ groups are independently chosen from C₁₋₄ alkyl or C₁₋₄ sulfoalkyl, most preferably ethyl or C₃₋₄ sulfoalkyl;
(ii) cyclised to give a cyclic Y¹ substituent of Formula Y^{c} or Y^{d}, more preferably of Formula Y^{c}.

The open chain form (i) is most preferred.

Especially preferred dyes of Formula III are of Formula IIIc, IIId or IIIe:

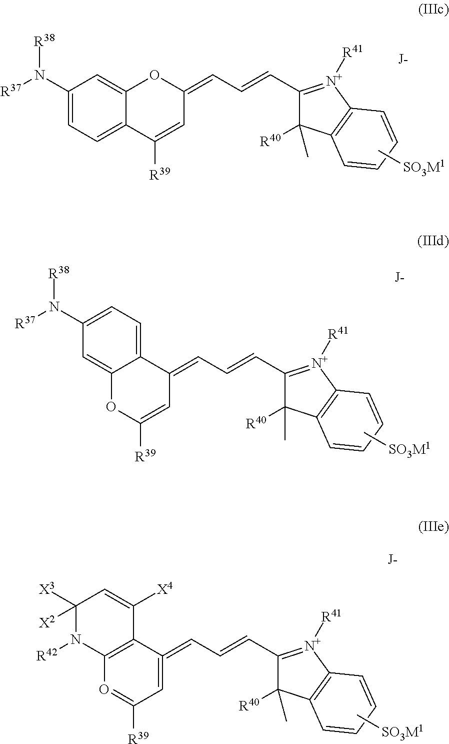

where:
M¹ and J are as defined above;
R³⁷ and R³⁸ are independently chosen from C₁₋₄ alkyl or C₁₋₄ sulfoalkyl;
R³⁹ is H or C₁₋₄ alkyl;
R⁴⁰ is C₁₋₄ alkyl, C₁₋₄ sulfoalkyl or C₁₋₆ carboxyalkyl;
R⁴¹ is C₁₋₄ sulfoalkyl or C₁₋₆ carboxyalkyl;
R⁴² is C₁₋₄ alkyl, C₁₋₄ sulfoalkyl or C₁₋₆ carboxyalkyl;
X², X³ and X⁴ are independently H or C₁₋₄ alkyl.

The dyes of Formulae IIId, IIIe and IIIf are preferably chosen such that one or more of R⁴⁰-R⁴² is C₁₋₄ sulfoalkyl.

Preferred specific dyes of Formula IIId are DY-631 and DY-633:

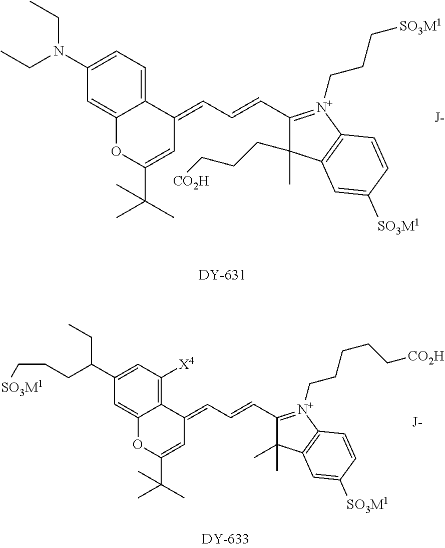

A preferred specific dye of Formula IIIe is DY-652:

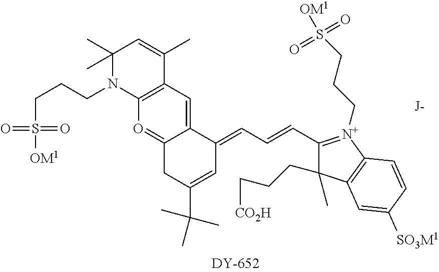

Preferred specific dyes are DY-631 and DY-652, with DY-652 being most preferred.

When present, it is envisaged that one of the roles of the linker group -(A)ₘ- is to distance the optical reporter from the active site of the vector. This is particularly important when the reporter is relatively bulky, so adverse steric interactions are possible. This can be achieved by a combination of flexibility (eg. simple alkyl chains), so that the reporter has the freedom to position itself away from the active site and/or rigidity such as a cycloalkyl or aryl spacer which orientate the reporter away from the active site. The nature of the linker group can also be used to modify the biodistribution of the imaging agent. Thus, eg. the introduction of ether groups in the linker will help to minimise plasma protein binding. When -(A)ₘ- comprises a polyethyleneglycol (PEG) building block or a peptide chain of 1 to 10 amino acid residues, the linker group may function to modify the pharmacokinetics and blood clearance rates of the imaging agent in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, eg. via the kidneys as opposed to via the liver.

By the term “sugar” is meant a mono-, di- or tri-saccharide. Suitable sugars include: glucose, galactose, maltose, mannose, and lactose. Optionally, the sugar may be functionalised to permit facile coupling to amino acids. Thus, eg. a glucosamine derivative of an amino acid can be conjugated to other amino acids via peptide bonds. The glucosamine derivative of asparagine (commercially available from NovaBiochem) is one example of this:

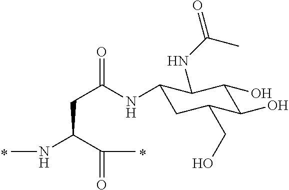

When a synthetic linker group (L) is present, it preferably comprises terminal functional groups which facilitate conjugation to the vector an optical reporter. When L comprises a peptide chain of 1 to 10 amino acid residues, the amino acid residues are preferably chosen from glycine, lysine, arginine, aspartic acid, glutamic acid or serine. When L comprises a PEG moiety, it preferably comprises units derived from oligomerisation of the monodisperse PEG-like structures of Formulae Bio1 or Bio2:

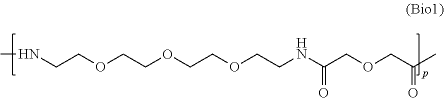

17-amino-5-oxo-6-aza-3,9,12,15-tetraoxaheptadecanoic acid of Formula Bio1 wherein p is an integer from 1 to 10. Alternatively, a PEG-like structure based on a propionic acid derivative of Formula Bio2 can be used:

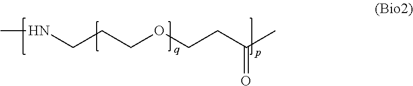

where p is as defined for Formula Bio1 and q is an integer from 3 to 15.

In Formula Bio2, p is preferably 1 or 2, and q is preferably 5 to 12.

When the linker group does not comprise PEG or a peptide chain, preferred L groups have a backbone chain of linked atoms which make up the -(A)ₘ- moiety of 2 to 10 atoms, most preferably 2 to 5 atoms, with 2 or 3 atoms being especially preferred. A minimum linker group backbone chain of 2 atoms confers the advantage that the optical reporter is well-separated so that any undesirable interaction is minimised.

The imaging agent of the first aspect is preferably provided as a pharmaceutical composition. Such compositions comprise the imaging agent, together with a biocompatible carrier, in a form suitable for mammalian administration. The “biocompatible carrier” is a fluid, especially a liquid, in which the imaging agent can be suspended or dissolved, such that the composition is physiologically tolerable, ie. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). Preferably the biocompatible carrier is pyrogen-free water for injection or isotonic saline.

The imaging agent and biocompatible carrier are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may optionally contain additional excipients such as an antimicrobial preservative, pH-adjusting agent, filler, stabiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The pharmaceutical compositions may be prepared under aseptic manufacture (ie. clean room) conditions to give the desired sterile, non-pyrogenic product. It is preferred that the key components, especially the associated reagents plus those parts of the apparatus which come into contact with the imaging agent (eg. vials) are sterile.

The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

The pharmaceutical compositions may also be prepared from a kit, as described in the fourth aspect (below).

The method of the first aspect is preferably used to distinguish:
(a) Barrett's oesophagus without dysplasia;
(b) dysplasia;
(c) carcinoma;

as described below. The determination would be via the fluorescent intensity. The method is primarily used to distinguish Barrett's oesophagus without dysplasia from dysplasia or carcinoma; preferably to distinguish each of (a), (b) and (c), and most preferably to further distinguish ‘low grade’ or ‘high grade’ dysplasia within (b)—where those terms are in relation to FIG. 1 (above).

The method of the first aspect preferably further comprises the step: (v) carrying out biopsy on the potential site(s) of dysplasia from step (iv). In this way, tissue samples can be taken for detailed testing ex vivo to confirm the nature and degree of any disease. The biopsy would be carried by standard techniques [see eg. Barr et al, Med Gen Med, 8, 66-88 (2006)].

With or without the biopsy step, the method of the first aspect is preferably used in the guidance of sites to deliver localised therapy. More accurate knowledge on the nature and degree of any disease permits the determination of the most appropriate therapy for the individual patient. Such therapy could include (see FIG. 1): chemotherapy with proton pump inhibitors, endoscopic mucosal ablation or surgical resection. If disease is detected and therapy is initiated, the method of the first aspect can advantageously be used the monitoring of the efficacy of therapy, eg. by follow-up repeat imaging at appropriate time intervals.

Optical reporters can be conjugated to vectors by conventional methods—see Achilefu [Technol. Cancer. Res. Treat., 3, 393-409 (2004)], Li et al [Org. Lett., 8(17), 3623-26 (2006) and Bullok et al, [J. Med. Chem., 48, 5404-5407 (2005)]. General methods for conjugation of cyanine dyes to biological molecules are described by Licha et al [Topics Curr. Chem., 222, 1-29 (2002); Adv. Drug Deliv. Rev., 57, 1087-1108 (2005)]. Peptide, protein and oligonucleotide vectors for use in the invention may be labelled at a terminal position, or alternatively at one or more internal positions. For reviews and examples of protein labelling using fluorescent dye labelling reagents, see “Non-Radioactive Labelling, a Practical Introduction”, Garman, A. J. Academic Press,1997; “Bioconjugation—Protein Coupling Techniques for the Biomedical Sciences”, Aslam, M. and Dent, A., Macmillan Reference Ltd, (1998). Protocols are available to obtain site specific labelling in a synthesised peptide, for example, see Hermanson, G. T., “Bioconjugate Techniques”, Academic Press (1996).

Methods of conjugating suitable optical reporters, in particular dyes, to amino acids and peptides are described by Licha (vide supra), as well as Flanagan et al [Bioconj. Chem., 8, 751-756 (1997)]; Lin et al, [ibid, 13, 605-610 (2002)] and Zaheer [Mol. Imaging, 1(4), 354-364 (2002)]. Methods of conjugating the linker group (L) to the vector use analogous chemistry to that of the dyes alone, and are known in the art.

Optical reporter dyes functionalised suitable for conjugation to peptides are commercially available from GE Healthcare Limited, Atto-Tec, Dyomics, Molecular Probes and others. Most such dyes are available as NHS (N-hydroxy succinimide) activated esters. Benzopyrylium dyes (Bzp^{M}) functionalised suitable for conjugation to vectors are commercially available from Dyomics (Dyomics GmbH, Winzerlaer Str. 2A, D-07745 Jena, Germany; www.dyomics.com), where the reactive functional group is NHS ester, maleimide, amino or carboxylic acid.

In a second aspect, the present invention provides specific imaging agents useful in the method of the first aspect, where said imaging agent is chosen from:
(i) hEGF or fragment thereof labelled with an optical reporter which is a cyanine dye of Formula Ia or a benzopyrylium dye;
(ii) TGF-alpha or fragment thereof labelled with an optical reporter which is a cyanine dye of Formula Ia or a benzopyrylium dye;
(iii) amphiregulin labelled with an optical reporter as defined in the first aspect;
(iv) a 9-20-mer peptide containing the amino acid sequence CKSPEPQHC (GE9), said peptide being labelled with an optical reporter which is a cyanine dye or a benzopyrylium dye;
(v) a 12-20-mer peptide containing the amino acid sequence LHLWVPEPWTQT (GE10) or YHWYGYTPQNVI (GE11), said peptide being labelled with an optical reporter which is a cyanine dye or a benzopyrylium dye;
(vi) a 12-20-mer peptide containing the amino acid sequence MLYNPTTYQMDVNPEGK (Inherbin 1), said peptide being labelled with an optical reporter as defined in the first aspect;
(vii) a 12-20-mer peptide containing the amino acid sequence LVYNKLTFQLEPNPHTK (Inherbin 3), said peptide being labelled with an optical reporter as defined in the first aspect;
(viii) an Affibody™ as defined in the first aspect, labelled with an optical reporter which is a cyanine dye of Formula Ia or a benzopyrylium dye;
(ix) a Nanobody™ as defined in the first aspect, labelled with an optical reporter which is a cyanine dye or a benzopyrylium dye.

Preferred aspects of the optical reporter of the imaging agent of the second aspect are as defined in the first aspect (above). In particular the vector of (i)-(vii) is preferably labelled with an optical reporter which is a cyanine dye of Formula Ia or a benzopyrylium dye. Preferred aspects of the cyanine dye and of the benzopyrylium dye are as defined in the first aspect.

In a third aspect, the present invention provides a pharmaceutical composition which comprises the imaging agent of the second aspect. The pharmaceutical composition, and preferred embodiments thereof, is as described in the first aspect (above). Preferred aspects of the pharmaceutical composition of the third aspect are as described in the second aspect.

In a fourth aspect, the present invention provides a kit of the preparation of the pharmaceutical composition of the third aspect. Suitable kits comprise the imaging agent of the first aspect in sterile, solid form such that, upon reconstitution with a sterile supply of a biocompatible carrier, dissolution occurs to give the desired pharmaceutical composition.

In that instance, the imaging agent, plus other optional excipients as described above, may be provided as a lyophilised powder in a suitable vial or container. The agent is then designed to be reconstituted with the desired biocompatible carrier to give the pharmaceutical composition in a sterile, apyrogenic form which is ready for mammalian administration. The term “biocompatible carrier” and preferred embodiments thereof, are as described in the first aspect (above).

A preferred sterile, solid form of the imaging agent is a lyophilised solid. The sterile, solid form is preferably supplied in a pharmaceutical grade container, as described for the pharmaceutical composition (above). When the kit is lyophilised, the formulation may optionally comprise a cryoprotectant chosen from a saccharide, preferably mannitol, maltose or tricine.

In a fifth aspect, the present invention provides the use of the imaging agent as defined in the first or second aspects in the method of in vivo imaging of the first aspect. Preferred aspects of the imaging agent for this use are as described in the first and second aspects.

In a sixth aspect, the present invention provides the use of a kit for the preparation of the imaging agent of the first aspect in the method of the first aspect.

In a seventh aspect, the present invention provides the use of the vector as defined in step (i) of the first aspect, in the manufacture of an imaging agent for use in the method of the first aspect.

The invention is illustrated by the following Examples. Compounds of the invention are shown in Table 2 (below). Compounds 1-3 are GE11 analogues. Compounds 4-7 are TGFα₂₂₋₃₁ fragment analogues, and Compounds 8-11 are EGF₂₁₋₃₁ fragment analogues.

Example 1 provides the synthesis of Cy5**, a preferred cyanine dye of the invention. Example 2 provides the synthesis of an activated ester of Cy5**. Example 3 provides the syntheses of peptides of the invention having conjugated thereto cyanine dye (Cy5**). Example 4 provides the syntheses of proteins of the invention having conjugated thereto cyanine dye (Cy5**), including an anti-EGFR Affibody. Example 5 provides the HPLC purification conditions for Compounds 1 to 14, and Example 6 the characterisation of Compounds 1 to 14. Example 7 provides binding assay data on the compounds of the invention, and shows that the vectors of the invention labelled with suitable optical reporters still retain binding to EGFR.

Example 8 provides data on the expression of EGFR in patient tissue samples from Barrett's oesophagus patients obtained via a tissue microarray method. Example 8 demonstrates that EGFR expression is low in Barrett's tissue and increases progressively from low grade dysplasia to high grade dysplasia to adenocarcinoma. EGFR expression is also increased in Barrett's tissue adjacent to sites of dysplasia/adenocarcinoma. EGFR expression in squamous tissue does not affect suitability of this target. That is because squamous tissue is the normal lining of the oesophagus, and Barrett's is the real background since that occupies a wide surface area; and is easily distinguished from squamous tissue under white light (because Barrett's tissue is pink, and thus easy to distinguish). The results show a significant upregulation of EGFR expression in “at risk” patients (patients that have progressed beyond Barrett's) and support EGFR targeting for biopsy guidance. The finding is the increased expression of EGFR in dysplasia.

Example 9 shows a trend for lower survival among high EGFR expressing patients in oesophageal cancer. This demonstrates the significance of EGF as a marker in that disease state.

### ABBREVIATIONS

Conventional single letter or 3-letter amino acid abbreviations are used.

Acm: Acetamidomethyl,

ACN: Acetonitrile,

Boc: tert-Butyloxycarbonyl,

Cy5**: see Example 1;

DBU: 1,8-Diazabicyclo[5.4.0]-undec-7-ene,

DCM: Dichloromethane,

DIEA; diisopropylethylamine,

DCC: N,N′-dicyclohexyl-carbodiimide,

DMSO: Dimethylsulfoxide,

Fmoc: 9-Fluorenylmethoxycarbonyl,

HBTU: O-Benzotriazol-1-yl-N,N,N′,N′-tetramethyluronium hexafluorophosphate

HOBt: 1-hydroxy-benzotriazole,

HSPyU: O-(N-succinimidyl)-N,N,N′,N′-tetramethyleneuronium hexafluorophosphate,

HPLC: High performance liquid chromatography,

ivDde: 1-(4,4-Dimethyl-2,6-dioxocyclohexylidene)-3-methylbutyl,

LC-MS: Liquid chromatography mass spectroscopy,

NHS: N-hydroxy-succinimide,

NMM: N-Methylmorpholine,

NMP: 1-Methyl-2-pyrrolidinone,

Pbf: 2,2,4,6,7-Pentamethyldihydrobenzofuran-5-sulfonyl,

PyAOP: 7-Azabenzotriazol-1-yloxy-tris-(pyrrolidino)phosphonium hexafluorophosphate,

rhEGF: recombinant human EGF;

rhTGFα: recombinant human TGFα;

RP-HPLC: reversed phase HPLC,

tBu: tertiary-butyl,

TFA: Trifluoroacetic acid,

TIS: Triisopropylsilane,

Trt: Trityl.

TABLE 2
Compounds of the Invention.

| Comp | Sequence | |
|---|---|---|
| 1 | a | H-Tyr-His-Trp-Tyr-Gly-Tyr-Thr-Pro-Gln-Asn-Val-Ile-NH₂ |
| | b | Cy5**-Tyr-His-Trp-Tyr-Gly-Tyr-Thr-Pro-Gln-Asn-Val-Ile-NH₂ |
| 2 | a | Fmoc-Tyr-His-Trp-Tyr-Gly-Tyr-Thr-Pro-Gln-Asn-Val-Ile-Lys-NH₂ |
| | b | H-Tyr-His-Trp-Tyr-Gly-Tyr-Thr-Pro-Gln-Asn-Val-Ile-Lys(Cy5**)-NH₂ |
| 3 | a | Fmoc-Tyr-His-Trp-Tyr-Gly-Tyr-Thr-Pro-Gln-Asn-Val-Ile-PEG-Lys-NH₂ |
| | b | H-Tyr-His-Trp-Tyr-Gly-Tyr-Thr-Pro-Gln-Asn-Val-Ile-PEG-Lys(Cy5**)-NH₂ |
| 4 | a | H-Arg-Phe-Leu-Val-Gln-Glu-Asp-Ser-Pro-Ala-OH |
| | b | Cy5**-Arg-Phe-Leu-Val-Gln-Glu-Asp-Ser-Pro-Ala-OH |
| 5 | a | ClCH₂CO-Arg-Phe-Leu-Val-Gln-Glu-Asp-Ser-Pro-Ala-Cys-Lys-NH₂ |
| | b | cyclo-(CH₂CO-Arg-Phe-Leu-Val-Gln-Glu-Asp-Ser-Pro-Ala-Cys)-Lys-NH₂ |
| | c | cyclo-(CH₂CO-Arg-Phe-Leu-Val-Gln-Glu-Asp-Ser-Pro-Ala-Cys)-Lys(Cy5**)-NH₂ |
| 6 | a | H-Arg-Phe-Leu-Val-Gln-Glu-Asp-Lys-Pro-Ala-Cys-NH₂ |
| | b | H-Arg-Phe-Leu-Val-Gln-Glu-Asp-Lys-Pro-Ala-Cys(Cy5**)-NH₂ |
| 7 | a | ClCH₂CO-Arg-Phe-Leu-Val-Gln-Glu-Asp-Lys-Pro-Ala-Cys-Lys-NH₂ |
| | b | cyclo-(CH₂CO-Arg-Phe-Leu-Val-Gln-Glu-Asp-Lys-Pro-Ala-Cys)-Lys-NH₂ |
| | c | cyclo-(CH₂CO-Arg-Phe-Leu-Val-Gln-Glu-Asp-Lys-Pro-Ala-Cys)-Lys(Cy5**)-NH₂ |
| 8 | a | H-Met-Tyr-Ile-Glu-Ala-Leu-Asp-Ser-Tyr-Asp-Cys-OH |
| 9 | a | ClCH₂CO-Met-Tyr-Ile-Glu-Ala-Leu-Asp-Ser-Tyr-Asp-Cys-Lys-NH₂ |
| | b | cyclo-(CH₂CO-Met-Tyr-Ile-Glu-Ala-Leu-Asp-Ser-Tyr-Asp-Cys)-Lys-NH₂ |
| | c | cyclo-(CH₂CO-Met-Tyr-Ile-Glu-Ala-Leu-Asp-Ser-Tyr-Asp-Cys)-Lys(Cy5**)-NH₂ |
| 10 | a | H-Met-Tyr-Ile-Glu-Ala-Leu-Asp-Lys-Tyr-Ala-Cys-NH₂ |
| | b | H-Met-Tyr-Ile-Glu-Ala-Leu-Asp-Lys-Tyr-Ala-Cys(Cy5**)-NH₂ |
| 11 | a | ClCH₂CO-Met-Tyr-Ile-Glu-Ala-Leu-Asp-Lys-Tyr-Ala-Cys-Lys-NH₂ |
| | b | cyclo-(CH₂CO-Met-Tyr-Ile-Glu-Ala-Leu-Asp-Lys-Tyr-Ala-Cys)-Lys-NH₂ |
| | c | cyclo-(CH₂CO-Met-Tyr-Ile-Glu-Ala-Leu-Asp-Lys-Tyr-Ala-Cys)-Lys(Cy5**)-NH₂ |
| 12 | | Cy5**-rhEGF |
| 13 | | Cy5**-rhTGFα |
| 14 | | anti-EGFR Affibody His₆-Z_{EGFR:1907}-Cys(Cy5**) |

### EXAMPLE 1

### Synthesis of the Cyanine Dye 2-{(1E,3E,5E)-5-[1-(5-carboxypentyl)-3,3-dimethyl-5-sulfo-1,3-dihydro-2H-indol-2-yliden]penta-1,3-dienyl}-3-methyl-1,3-bis(4-sulfobutyl)-3H-indolium-5-sulfonate (Cy5**)

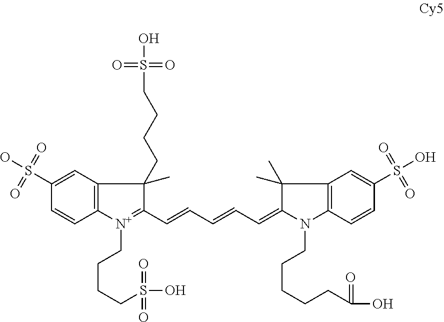

### (1a) 5-Methyl-6-oxoheptane-1-sulfonic Acid

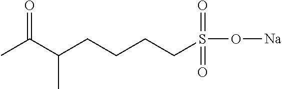

Ethyl 2-methylacetoacetate (50 g) in DMF (25 ml) was added to a suspension of sodium hydride (12.0 g of 60% NaH in mineral oil) in DMF (100 ml), dropwise with ice-bath cooling over 1 hour, (internal temperature 0-4° C.). This mixture was allowed to warm to ambient temperature for 45 mins with stirring before re-cooling. A solution of 1,4-butanesultone (45 g) in DMF (25 ml) was then added dropwise over 15 minutes. The final mixture was heated at 60° C. for 18 hours. The solvent was removed by rotary evaporation and the residue partitioned between water and diethyl ether. The aqueous layer was collected, washed with fresh diethyl ether and rotary evaporated to yield a sticky foam. This intermediate was dissolved in water (100 ml) and sodium hydroxide (17.8 g) added over 15 minutes with stirring. The mixture was heated at 90° C. for 18 hours. The cooled reaction mixture was adjusted to ˜pH2 by the addition of concentrated hydrochloric acid (˜40 ml). The solution was rotary evaporated and dried under vacuum. The yellow solid was washed with ethanol containing 2% hydrochloric acid (3×150 ml). The ethanolic solution was filtered, rotary evaporated and dried under vacuum to yield a yellow solid. Yield 70 g.

### (1b) 2,3-Dimethyl-3-(4-sulfobutyl)-3H-indole-5-sulfonic acid, dipotassium Salt

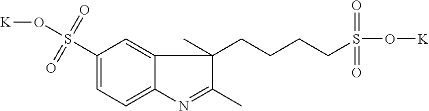

4-Hydrazinobenzenesulfonic acid (40 g), 5-methyl-6-oxoheptane-1-sulfonic acid (from 1a; 60 g) and acetic acid (500 ml) were mixed and heated under reflux for 6 hrs. The solvent was filtered, rotary evaporated and dried under vacuum. The solid was dissolved in methanol (1 L). To this was added 2M methanolic potassium hydroxide (300 ml). The mixture was stirred for 3 hours and then the volume of solvent reduced by 50% using rotary evaporation. The resulting precipitate was filtered, washed with methanol and dried under vacuum. Yield 60 g. MS (LCMS): MH⁺ 362. Acc. Mass: Found, 362.0729. MH⁺═C₁₄H₂₀NO₆S₂ requires m/z 362.0732 (−0.8 ppm).

### (1c) 2,3-Dimethyl-1,3-bis(4-sulfobutyl)-3H-indolium-5-sulfonate, dipotassium Salt

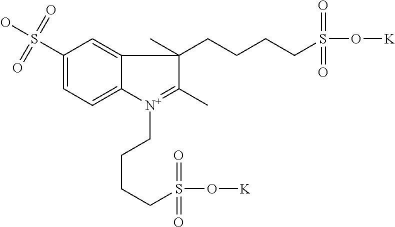

2,3-Dimethyl-3-(4-sulfobutyl)-3H-indole-5-sulfonic acid (from 1b; 60 g) was heated with 1,4 butane sultone (180 g) and tetramethylene sulfone (146 ml) at 140° C. for 16 hours. The resulting red solid was washed with diethyl ether, ground into a powder and dried under vacuum. Yield 60 g.

### (1d) Cy5**, as TFA Salt

1-(5′-Carboxypentyl)-2,3,3-trimethyl-indolenium bromide-5-sulfonic acid, K⁺ salt (2.7 g), malonaldehyde bis(phenylimine)monohydrochloride (960 mg), acetic anhydride (36 ml) and acetic acid (18 ml) were heated at 120° C. for 1 hour to give a dark brown-red solution. The reaction mixture was cooled to ambient temperature. 2,3-Dimethyl-1,3-bis(4-sulfobutyl)-3H-indolium-5-sulfonate (from 1c; 8.1 g) and potassium acetate (4.5 g) were added to the mixture, which was stirred for 18 hours at ambient temperature. The resulting blue solution was precipitated using ethyl acetate and dried under vacuum. The crude dye was purified by liquid chromatography (RPC₁₈. Water+0.1% TFA/MeCN+0.1% TFA gradient). Fractions containing the principal dye peak were collected, pooled and evaporated under vacuum to give the title dye, 2 g. UV/Vis (Water+0.1% TFA): 650 nm. MS (MALDI-TOF): MH+ 887.1. MH⁺═C₃₈H₅₀N₂O₁₄S₄ requires m/z 887.1.

### EXAMPLE 2

### Synthesis of 2-[(1E,3E,5E)-5-(1-{6-[(2,5-dioxopyrrolidin-1-yl)oxy]-6-oxohexyl}-3,3-dimethyl-5-sulfo-1,3-dihydro-2H-indol-2-ylidene)penta-1,3-dienyl]-3-methyl-1,3-bis(4-sulfobutyl)-3H-indolium-5-sulfonate, diisopropylethylamine Salt (NHS Ester of Cy5**)

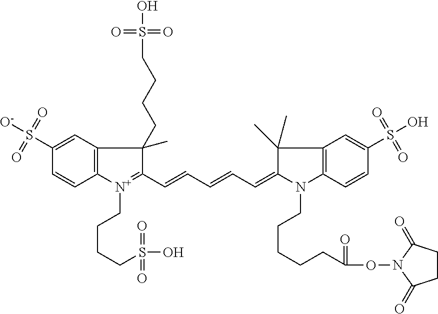

Cy5** (Example 1, 10 mg) was dissolved in anhydrous DMSO (3 ml); to this were added HSPyU (20 mg) and N,N′-diisopropylethylamine (80 μl). The resulting solution was mixed for 3 hours, whereupon TLC (RPC18. Water/MeCN) revealed complete reaction. The dye was isolated by precipitation in ethyl acetate/diethyl ether, filtered, washed with ethyl acetate and dried under vacuum. UV/Vis (Water) 650 nm. MS (MALDI-TOF) MH+983.5. MH⁺═C₄₂H₅₃N₃O₁₆S₄ requires m/z 984.16.

### EXAMPLE 3

### Synthesis of Compounds 1-11

a) Peptide Synthesis

The peptidyl resin corresponding to the sequences of Compounds 1-11 in Table 2 were assembled by standard solid-phase peptide chemistry [Barmy, Int. J. Peptide Protein Res., 30, 705-739 (1987)] on either a Rink Amide MBHA resin (from NovaBiochem, typical loading 0.72 mmol/g, synthesis of 1, 2, 3, 5, 6, 7, 9, 10 and 11), a Fmoc-Ala-Wang resin (from NovaBiochem, loading 0.52 mmol/g, synthesis of 4) or on a Fmoc-Cys(Trt)-Sasrin resin (from Bachem, loading 0.45 mmol/g, synthesis of 8). Fmoc-Cys(Trt)-OH was loaded manually onto the Rink Amide MBHA resin using PyAOP/collidine activation in 50% DCM:DMF (synthesis of 6 and 10). All other amino acids were assembled manually on the solid phase using a microwave assisted peptide synthesizer (CEM Liberty). The residues (from the carboxyl terminus) were coupled on a 0.1 mmol scale typically using single coupling cycles (5 min at 75° C.) of 0.5 mmol Fmoc-amino acids (0.2 M solution in NMP activated by adding 0.45 mmol HBTU and HOBt (0.45M solution of both reagents in DMF), followed by 1 mmol DIEA (2M solution in NMP). The His and Arg residues were incorporated using a double coupling procedure. Fmoc-deprotection was achieved with 20% piperidine in NMP. The washing solvent was NMP. The polyethyleneglycol (PEG) residue used, Fmoc-amino PEG diglycolic acid, was from Polypure (synthesis of 3). All other amino acids were from CEM. The amino acid-side chain protecting groups used were either tBu for Asp, Glu, Ser, Thr and Tyr; Boc for Lys, Trp and His; trityl for Asn, Cys and Gln; Pbf for Arg; and ivDde for Lys²⁹ in 7 and for Lys²⁸ in 11.

b) Chloroacetylation of Peptide Resin Precursors of Compounds 5a, 7a, 9a and 11a.

The peptide resin precursors of 5a, 7a, 9a, and 11a were chloroacetylated on a 0.1 mmol scale. Chloroacetic acid (5 eq) and DCU (2.5 eq) were dissolved in DCM (5 mL) and the mixture was stirred for 30 min. The solid urea formed was removed by filtration and the filtrate evaporated to dryness under reduced pressure. The residue (symmetrical anhydride) was dissolved in NMP (5 mL) and added to the peptide resins contained in a nitrogen bubbler apparatus. Complete reaction was confirmed after 2 h by a negative Kaiser test.

c) Deprotection and Cleavage from the Resin.

The peptide resins were treated with TFA containing 2.5% TIS and 2.5% water for 2 hours, using a manual nitrogen bubbler apparatus [Wellings, D. A., Atherton, E. (1997) in Methods in Enzymology (Fields, G. Ed), 289, p. 53-54, Academic Press, New York]. Ethanedithiol was an additional component of the TFA cleavage mixture in the case of 8, 9, 10 and 11. Under these conditions the peptides were cleaved from the resins whilst simultaneously removing all side-chain protecting groups from the peptide, except for the ivDde and Fmoc groups. The cleavage mixtures were filtered and washed with small quantities of neat TFA. The combined filtrate and washings were concentrated by rotary evaporation and then triturated with diethyl ether to obtain the crude peptides. The precipitates were isolated by centrifugation, washed with ether and then lyophilized from 50% ACN-0.1% aq. TFA yielding the crude products. Compound 1a, 2a and 3a containing Trp residues were stirred overnight in 50% ACN-water prior to lyophilization in order to complete the Trp-deprotection step.

d) Cyclization of Compounds 5a, 7a, 9a and 11a.

The peptides were dissolved in degassed 60% ACN-water, and the pH adjusted to 7.5-8 with 25% ammonia-water. The cyclization was allowed to proceed under a blanket of argon for 2.5 hours and the mixture was acidified and lyophilized affording 5b, 7b, 9b and 11b.

e) Cy5** Conjugation.

Compounds 1a, 2a, 3a, 4a, 5b, 7b, 8a, 9b and 11b were conjugated to Cy5** either at the N^{α} amino group (1a, 4a, 8a) or at the N^{ε}-amino group of the C-terminal Lys side-chain residue (2a, 3a, 5b, 7b, 9b and 11b). Cy5** (Example 1, 3 eq) was dissolved in DMF (1 mL) and solid HBTU (2.7 eq) was added followed by NMM or DIEA (20 eq) and let react for about 10-25 min. The pre-activated dye was added to the solid peptides (0.01 mmol). LC-MS monitoring indicated complete conjugation after 1.5-2 hours and the products purified by preparative RP-HPLC (conditions below) affording pure 1b, 4b, 5c and 9c. Pure 2b and 3b were obtained after subsequent Fmoc-deprotection with DBU (5 eq) and N-(2-mercaptoethyl)-aminomethyl polystyrene resin (10 eq) in DMF (1.5 mL) for 1 hour and final RP-HPLC purification (conditions below). Pure 7c and 11c were obtained after subsequent ivDde-deprotection with 2% hydrazine in DMF (1 mL) for 15 min followed by acidification and final RP-HPLC purification (conditions below).

Compounds 6a and 10a were conjugated to Cy5** at the thiol group of the C-terminal Cys side-chain residue. Tetra-sulfonated cyanine dye Cy5**-N-(2-aminoethyl)maleimide was used, prepared by activating the corresponding free acid (1.1 eq) with HBTU (1 eq) and sym. collidine (5 eq) in DMF and reacting the pre-activated dye with N-(2-Aminoethyl)maleimide followed by preparative RP-HPLC purification. Solutions of 6a and 10a (0.004 mmol, 1 eq) and tris(2-carboxyethyl)phosphine hydrochloride (TCEP) (5 eq) in DMF-0.05 M PBF buffer (1:2, 3 mL) were added to solid Cy5**-N-(2-Aminoethyl)maleimide (3 eq). LC-MS monitoring indicated complete conjugations after 1.5 hours and the reaction mixtures were purified by preparative RP-HPLC.

f) Purification.

The crude peptides were purified by preparative RP-HPLC (Beckman System Gold chromatography systems). The column (Phenomenex Luna C18 5μ, 22×250 mm) was eluted at 10 mL/min using gradients over 40, 60 or 80 min (Table 2). The eluents used were water containing 0.1% TFA, 0.05% TFA or 0.1% formic acid (FA) (solvent A) and ACN containing 0.1% TFA, 0.05% TFA or 0.1% FA (solvent B). The eluent was monitored at μ=214 nm. The desired peak fractions were pooled and lyophilized affording pure products. See the conditions used for the specific compounds in Table 3.

g) Characterization.

The compounds were characterised by LC-MS (Table 4). LC-MS data was recorded on one of three different instruments: 1) a LCQ DECA XP MAX instrument (Thermo Finnigan) using electrospray ionization (ESI), operated in positive mode at 4.5 kV with a scan rate 5500 Da/s and coupled to Thermo Finnigan Surveyor PDA chromatography system run under the following conditions: Solvent A=water/0.1% TFA, 0.05% TFA or 0.1% FA and solvent B=ACN/0.1% TFA, 0.05% TFA or 0.1% FA using 5 min gradients; flow rate: 0.6 mL/min; column: Phenomenex Luna 3 μm C18 (2) 20×2 mm; detection: PDA. 2) a Quatro Premier instrument (Micromass Technologies/Waters UK) using ESI operated in a positive mode at 3.0 kV with a scan rate of 2000 Da/s, coupled to a Waters chromatography system equipped with a 1525□ binary HPLC pump, a 2777c Sample Manager and a 2996 PDA detector and run under the same conditions as described above. 3) A LCT Premier ESI time-of-flight instrument (Micromass Technologies/Waters UK) coupled to a Acquity Ultra Performance chromatography system (Waters) run under the following conditions: Solvent A and B as above using 10 min gradients; flow rate 0.5 ml/min; column: Zorbax SB-AQ 3.5 mm, 3×100 mm; PDA detection. Data acquisition and processing were performed using Xcaliber (instrument 1) or Masslynx 4.0 (instruments 2 and 3) data system. See Table 4 for specific conditions and results.

### EXAMPLE 4

### Synthesis of Compounds 12-14

a) Cy5**-Labelling of rhEGF (Compound 12).

Recombinant human EGF (rhEGF; ˜4 mg, PBS-salt containing ˜0.5 mg EGF, from R&D Systems) was dissolved in water (0.4 mL) and added to 0.1M NaHCO₃ (0.05 mL). Cy5**-NHS ester (Example 2) in water at 4 mg/mL was added (5 eq, 0325 mL) and the pH was adjusted to 8.3 with 0.1M NaHCO₃. LC-MS monitoring showed about 70-80% EGF consumption after about 2 h. The reaction mixture was at this point quenched by addition of 20% ACN-01% aq. TFA, and purified by RP-HPLC, see conditions described in Example 3f) and in Table 2. Fractions of the major peaks identified as mono-labelled rhEGF 12 by LC-MS analysis were lyophilized, see conditions described in Example 3g) and in Table 3.

b) Cy5**-Labelling of rhTGFα (Compound 13).

Recombinant human TGFα (rhTGFα; ˜0.5 mg, from R&D Systems) was dissolved in water (1.25 mg/mL, 0.4 mL, 1 eq), and 0.05 mL 0.1M NaHCO₃ was added to give a pH of ˜8.5. Cy5**-NHS ester in water (Example 2; 4 mg/mL, 0.1 mL, 5 eq) was added to the TGFα solution and the pH was adjusted to ˜8.2 with 0.1M NaHCO₃. LC-MS analysis after 90 min showed good reaction progress; about 60% conversion to a mono dye labelled product. Additional Cy5**-NHS ester (2.5 eq) was needed in order to drive the reaction close to completion after 3 h. The reaction was then quenched by addition of 20% ACN-01% aq. TFA and the product purified by preparative RP-HPLC, see conditions described in Example 3f) and in Table 3. Fractions of the major peak identified as desired mono-labelled rhTGFα 13 by LC-MS analysis were lyophilized, see conditions described in Example 3g) and in Table 4.

c) Cy5**-Labelling of Anti-EGFR Affibody His₆-ZEGFR: ₁₉₀₇-Cys.

The His₆-ZEGFR: ₁₉₀₇-Cys affibody was dissolved in 0.1M PBS buffer, pH 7.4 (0.2 ml) and a solution of TCEP in PBS-buffer (1 mg/mL, 0.2 mL) was added and the mixture was placed in the refrigerator for 2 h to ensure fully reduced Cys residues. A solution of tetra-sulfonated Cy5**-N-(2-Aminoethyl)maleimide (1.3 mg) in PBS-buffer (0.2 mL) was added and the reaction progress was monitored by LC-MS. After 6 h total conversion to the desired product was observed and the Cy5**-conjugated affibody was isolated by preparative RP-HPLC purification using 0.05% TFA based eluents, see conditions described in Example 3f) and in Table 3. 1M ammonium acetate buffer was added to the receiving vials to neutralize the eluent since the affibody is prone to aggregate at pH below 4.5. Fractions of the major peak identified as desired mono-labelled anti-EGFR affibody 14 by LC-MS analysis were lyophilized, see conditions described in Example 3g) and in Table 4.

### EXAMPLE 5

### RP-HPLC Purification of Compounds 1-14

The details are summarised in Table 3:

TABLE 3
RP-HPLC Purification

| | | | % acid in | Retention | |
|---|---|---|---|---|---|
| Compound | | Gradient | solvents | time (min) | Yield (mg) |
| 1 | a | 20-30% B; 80 min | 0.1 TFA | 38.4 | 5.3 |
| | b | 20-30% B; 80 min | 0.1 TFA | 49.5 | 7.6 |

| | | | | |
|---|---|---|---|---|
| 2b | 20-30% B; 80 min | 0.1 TFA | 46.4 | 4.6 |
| 3b | 20-30% B; 80 min | 0.1 TFA | 48.6 | 5.5 |
| 4b | 15-50% B; 40 min | 0.1 TFA | 20.9 | 5.4 |
| 5c | 18-25% B; 40 min | 0.1 TFA | 41.5 | 4.6 |
| 6b | 15-30% B; 40 min | 0.1 TFA | 29.8 | 6.0 |
| 7c | 20-40% B; 40 min | 0.1 FA | 31.3 | 1.2 |
| 9c | 20-50% B; 40 min | 0.1 TFA | 20.4 | 3.3 |
| 10b | 20-50% B; 40 min | 0.1 FA | 27.6 | 5.3 |
| 11c | 25-50% B; 40 min | 0.1 TFA | 21.4 | 1.6 |
| 12 | 20-40% B; 60 min | 0.1 TFA | 30.2 | 0.2-0.3 |
| 13 | 25-40% B; 60 min | 0.1 TFA | 30.8 | 0.2-0.3 |
| 14 | 30-70% B, 40 min | 0.05 TFA | 17.3 | 0.1-0.2 |

### EXAMPLE 6

### Characterisation of Compounds 1-14

The details are summarised in Table 4:

TABLE 4
Characterisation of Compounds 1-14.

| | | | % acid in | | | Retention | MS | |
|---|---|---|---|---|---|---|---|---|
| Compound | | Gradient | solvents | | Instr. | time | m/z expected | m/z [symbol] |
| 1 | a | 15-30% B | 0.1 | TFA | 2 | 2.9 | MH⁺ 1539.7 | 1540.2 |
| | b | 15-30% B | 0.1 | TFA | 1 | 3.6 | MH₂²⁺ | 1204.6 |
| 2 | b | 15-30% B | 0.1 | TFA | 2 | 3.2 | MH₂²⁺ | 1268.8 |
| 3 | b | 15-30% B | 0.1 | TFA | 2 | 3.3 | MH₂²⁺ | 1413.8 |
| 4 | b | 15-30% B | 0.1 | TFA | 1 | 3.1 | MH₂²⁺ | 1015.9 |
| 5 | c | 15-40% B | 0.1 | TFA | 1 | 1.9 | MH₂²⁺ | 1150.9 |
| 6 | b | 10-30% B | 0.1 | TFA | 1 | 3.3 | MH₂²⁺ | 1157.5 |
| 7 | c | 15-40% B | 0.1 | FA | 1 | 1.7 | MH₂²⁺ | 1171.5 |
| 8 | a | 15-25% B | 0.1 | FA | 1 | 3.1 | MH⁺ 1278.3 | 1278.5 |
| 9 | c | 10-40% B | 0.1 | TFA | 1 | 3.4 | MH₂²⁺ | 1157.9 |
| 10 | b | 20-50% B | 0.1 | FA | 1 | 2.0 | MH₂²⁺ | 1164.5 |
| 11 | c | 20-40% B | 0.1 | TFA | 1 | 1.4 | MH₂²⁺ | 1178.5 |
| 12 | | 20-40% B | 0.1 | TFA | 1 | 2.7 | MH₄⁴⁺ | 1804.6 |
| 13 | | 15-35% B | 0.05 | TFA | 3 | 7.7 | MH₄⁴⁺ | 1604.3 |
| 14 | | 2-95% B | 0.05 | TFA | 3 | 5.2 | MH₅⁵⁺ 1834.2 | 1833.8 |

[symbol]

indicates data missing or illegible when filed

### EXAMPLE 7

### Fluorescence Polarization Binding Assay

Compounds 12, 13 and 14 were tested in a fluorescence polarization binding assay towards human EGFR.

The fluorescence polarization binding measurements were performed as triplicates in 384-well microplates in a volume of 40 μL in binding buffer (PBS, 0.01% Tween-20, pH 7.5) using a Tecan Safire fluorescence polarisation plate reader at ex646/em678 nm. The concentration of dye-labelled ligand was held constant (5 nM) and the concentrations of the target (human recombinant EGFR/Fc chimera from R&D systems, cat. no 344-ER) was varied from 0-258 nM. Binding mixtures were equilibrated in the microplate for 10 min at 30° C. The observed change in anistropy was fitted to the equation

$${γ}_{obs} = {γ}_{free} + ( {γ}_{bound} - {γ}_{free} ) \frac{( Kd + cT + P ) - \sqrt{( Kd + cT + P ) 2 - 4 \cdot cT \cdot P}}{2 \cdot P}$$

where γ_{obs} is the observed anistropy, γ_{free} is the anistropy of the free peptide, γ_{bound} is the anistropy of the bound peptide, K_{d} is the dissociation constant, cT is the total target concentration, and P is the total Dye-labelled peptide concentration. The equation assumes that the ligand and the receptor form a reversible complex in solution with 1:1 stoichiometry. Data fitting was performed via nonlinear regression using SigmaPlot software (version 10) to obtain the K_{d} value.

The results show a Kd value of 11.9+/−1.4 nM and 22.1+/−3.7 nM for compound 12 and 13, respectively while compound 14 has a Kd value of 4.1+/−0.4 nM towards human EGFR.

### EXAMPLE 8

### Tissue Microarray

Fresh biopsy tissue samples were collected from Barrett's oesophagus patients, having a range of histologies. There were 107 samples in total, comprising: 20 gastric controls; 7 squamous controls; 20 Barrett's metaplasia; 20 low-grade dysplasia; 20 high-grade dysplasia; and 20 adenocarcinomas. Tissue Microarray was carried on the above tissue samples, where each core had a diameter of ˜0.6 mm with variable depth. A protocol for Tissue Microarray is described by Camp et al [“Validation of tissue microarray technology in breast carcinoma”. Lab. Invest. 80, 1943-1949 (2000)]. Full section slides were from the same paraffin blocks that the cores were obtained from. All tissue was from patient biopsies and no patients had received chemotherapy specifically for oesophageal dysplasia/adenocarcinoma.

The expression of the biological marker EGFR was determined via immunohistochemistry staining with an EGFR targeting antibody, followed by scoring on a 0-3 basis. Additionally, the same analysis was carried out in full slides, using a neg/+/++/+++/++++/+++++ scoring method. The results are described below. Metaplasia refers to an abnormal change in the nature of the tissue (in oesophageal metaplasia, normal squamous epithelium is replaced by columnar epithelium), whereas dysplasia refers to abnormal tissue growth (in oesophageal dysplasia, the metaplastic columnar epithelium begins to lose control of proliferation).

EGFR Expression.

EGFR expression in different stages of oesophageal carcinogenesis, was assessed by immunohistochemistry, using tissue arrays or full tissue slides. The results are shown in FIG. 2:

### EXAMPLE 9

### Kaplan-Meier Curve Analysis of EGFR Expression and Post-Diagnosis Patient Survival

Kaplan-Meier curve analysis was carried out using originating patient survival data (where available). Kaplan-Meier analysis is a well-known clinical tool for correlating a parameter with patient survival. Oesophageal cancer patients were studied over time to see whether survival correlated with individual expression of EGFR—low (green group) or high (blue group) EGFR levels. On day zero which was the first day of observation, all patients were alive in both groups. On day ˜300, in both groups 40% of patients were alive, i.e no effect of EGFR expression yet. By day ˜700, however, all patients expressing strong levels of EGFR had died, while patients with low EGFR expression had longer survival (the last one died ˜2 years later).

What is claimed is:

1-20. (canceled)

21. A method of in vivo imaging, for use in the determination of sites of potential dysplasia in a patient suffering from Barrett's oesophagus, said method comprising:
(i) provision of an imaging agent which comprises a vector which targets the extracellular domain of EGFR, said vector being labelled with an optical reporter imaging moiety suitable for imaging the mammalian body in vivo using light of wavelength 500-1200 nm;
(ii) carrying out optical imaging at least a portion of the oesophagus of said patient with the imaging agent from step (i);
(iii) making a determination from the imaging of step (ii) whether there is increased uptake of the imaging agent relative to background at one or more locations of the patient's oesophagus;
(iv) when the determination of step (iii) shows increased uptake for at least one such location, that location is identified as a site of potential dysplasia;
wherein said vector is chosen from:
(a) a 9-20-mer peptide containing the peptide sequence CKSPEPQHC (GE9);
(b) a 2-20-mer peptide containing the peptide sequence LHLWVPEPWTQT (GE10);
(c) a 12-20-mer peptide containing the peptide sequence YHWYGYTPQNVI (GE11);
(d) a 17-20-mer peptide containing the peptide sequence MLYNPTTYQMDVNPEGK (Inherbin 1);
(e) a 17-20-mer peptide containing the peptide sequence LVYNKLTFQLEPNPHTK (Inherbin 3);
(f) an Affibody™;
(g) a Nanobody™; and
(h) 6-15-mer peptides containing the peptide sequence LARLLT (D4).

22. The method of claim 21, where the optical reporter is a fluorescent dye.

23. The method of claim 22, where the fluorescent dye is a cyanine dye.

24. The method of claim 23, where the cyanine dye is of Formula Ia: 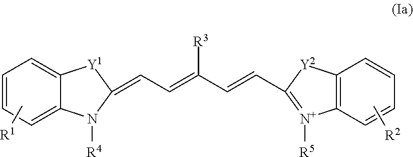
where:
Y¹ and Y² are independently —O—, —S—, —NR⁶— or —CR⁷R⁸— and are chosen such that at least one of Y¹ and Y² is —CR⁷R⁸—;
R¹ and R² are independently H, —SO₃M¹ or R^{a}, where M¹ is H or B^{c}, and B^{c} is a biocompatible cation;
R³ is H, C₁₋₅ alkyl, C₁₋₆ carboxyalkyl or an R^{a} group;
R⁴ to R⁶ are independently C₁₋₅ alkyl, C₁₋₆ carboxyalkyl or R^{a};
R⁷ is H or C₁₋₃ alkyl;
R⁸ is R^{a} or C₁₋₆ carboxyalkyl;
R^{a} is C₁₋₄ sulfoalkyl;
with the proviso that the cyanine dye of Formula Ia comprises at least one R^{a} group and a total of 1 to 6 sulfonic acid substituents from the R¹, R² and R^{a} groups.

25. The method of claim 22, where the dye is a benzopyrylium dye.

26. The method of claim 21, where the imaging agent of step (i) of claim 21 is provided as a pharmaceutical composition.

27. The method of claim 21, where the imaging is used to distinguish between:
(a) Barrett's oesophagus without dysplasia;
(b) dysplasia; and
(c) carcinoma.

28. The method of claim 21, further comprising the step:
(v) carrying out biopsy on the potential site(s) of dysplasia from step (iv).

29. An imaging agent chosen from:
(i) a 9-20-mer peptide containing the amino acid sequence CKSPEPQHC (GE9), said peptide being labelled with an optical reporter wherein said optical reporter is a fluorescent dye;
(ii) a 12-20-mer peptide containing the amino acid sequence LHLWVPEPWTQT (GE10) or YHWYGYTPQNVI (GE11), said peptide being labelled with an optical reporter wherein said optical reporter is a fluorescent dye;
(iii) a 12-20-mer peptide containing the amino acid sequence MLYNPTTYQMDVNPEGK (Inherbin 1), said peptide being labelled with an optical reporter wherein said optical reporter is a fluorescent dye;
(iv) a 12-20-mer peptide containing the amino acid sequence LVYNKLTFQLEPNPHTK (Inherbin 3), said peptide being labelled with an optical reporter wherein said optical reporter is a fluorescent dye;
(v) the Affibody™ labelled with an optical reporter wherein said optical reporter is selected from the group consisting of: a fluorescent dye, a cyanine dye of Formula Ia: 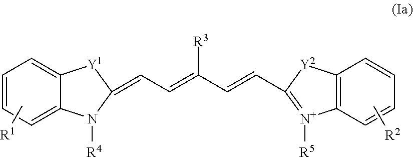
where:
Y¹ and Y² are independently —O—, —S—, —NR⁶— or —CR⁷R⁸— and are chosen such that at least one of Y¹ and Y² is —CR⁷R⁸—;
R¹ and R² are independently H, —SO₃M¹ or R^{a}, where M¹ is H or B^{c}, and B^{c} is a biocompatible cation;
R³ is H, C₁₋₅ alkyl, C₁₋₆ carboxyalkyl or an R^{a} group;
R⁴ to R⁶ are independently C₁₋₅ alkyl, C₁₋₆ carboxyalkyl or R^{a};
R⁷ is H or C₁₋₃ alkyl;
R⁸ is R^{a} or C₁₋₆ carboxyalkyl;
R^{a} is C₁₋₄ sulfoalkyl;
with the proviso that the cyanine dye of Formula Ia comprises at least one R^{a} group and a total of 1 to 6 sulfonic acid substituents from the R¹, R² and R^{a} groups; and
a benzopyrylium dye; and
(vi) a Nanobody™ labelled with an optical reporter wherein the optical reporter is is selected from the group consisting of: a fluorescent dye, a cyanine dye of Formula Ia: 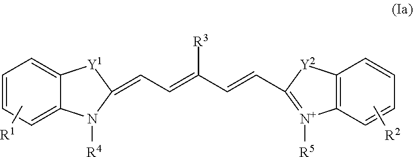
where:
Y¹ and Y² are independently —O—, —S—, —NR⁶— or —CR⁷R⁸— and are chosen such that at least one of Y¹ and Y² is —CR⁷R⁸—;
R¹ and R² are independently H, —SO₃M¹ or R^{a}, where M¹ is H or B^{c}, and B^{c} is a biocompatible cation;
R³ is H, C₁₋₅ alkyl, C₁₋₆ carboxyalkyl or an R^{a} group;
R⁴ to R⁶ are independently C₁₋₅ alkyl, C₁₋₆ carboxyalkyl or R^{a};
R⁷ is H or C₁₋₃ alkyl;
R⁸ is R^{a} or C₁₋₆ carboxyalkyl;
R^{a} is C₁₋₄ sulfoalkyl;
with the proviso that the cyanine dye of Formula Ia comprises at least one R^{a} group and a total of 1 to 6 sulfonic acid substituents from the R¹, R² and R^{a} groups; and
a benzopyrylium dye.

30. The imaging agent of claim 29, where the optical reporter is said cyanine dye of Formula Ia or said benzopyrylium dye.

31. A pharmaceutical composition which comprises the imaging agent of claim 29.

32. A kit for the preparation of the pharmaceutical composition of claim 31.


SEQUENCE LISTING (20 sequences)

SEQ ID NO: 1 - protein, 9 aa, Artificial
  synthetic peptide
CKSPEPQHC

SEQ ID NO: 2 - protein, 12 aa, Artificial
  synthetic peptide
LHLWVPEPWTQT

SEQ ID NO: 3 - protein, 12 aa, Artificial
  synthetic peptide
YHWYGYTPQNVI

SEQ ID NO: 4 - protein, 17 aa, Artificial
  synthetic peptide
MLYNPTTYQMDVNPEGK

SEQ ID NO: 5 - protein, 17 aa, Artificial
  Synthetic peptide
LVYNKLTFQLEPNPHTK

SEQ ID NO: 6 - protein, 6 aa, Artificial
  synthetic peptide
LARLLT

SEQ ID NO: 7 - protein, 12 aa, Artificial
  synthetic peptide
YHWYGYTPQNVI

SEQ ID NO: 8 - protein, 13 aa, Artificial
  synthetic peptide
YHWYGYTPQNVIK

SEQ ID NO: 9 - protein, 12 aa, Artificial
  synthetic peptide
YHWYGYTPQNVI

SEQ ID NO: 10 - protein, 10 aa, Artificial
  synthetic peptide
RFLVQEDSPA

SEQ ID NO: 11 - protein, 12 aa, Artificial
  synthetic peptide
RFLVQEDSPACK

SEQ ID NO: 12 - protein, 12 aa, Artificial
  synthetic peptide
RFLVQEDSPACK

SEQ ID NO: 13 - protein, 11 aa, Artificial
  synthetic peptide
RFLVQEDKPAC

SEQ ID NO: 14 - protein, 12 aa, Artificial
  synthetic peptide
RFLVQEDKPACK

SEQ ID NO: 15 - protein, 12 aa, Artificial
  synthetic peptide
RFLVQEDKPACK

SEQ ID NO: 16 - protein, 12 aa, Artificial
  synthetic peptide
MYIEALDSYDCK

SEQ ID NO: 17 - protein, 12 aa, Artificial
  synthetic peptide
MYIEALDSYDCK

SEQ ID NO: 18 - protein, 11 aa, Artificial
  synthetic peptide
MYIEALDKYAC

SEQ ID NO: 19 - protein, 12 aa, Artificial
  synthetic peptide
MYIEALDKYACK

SEQ ID NO: 20 - protein, 12 aa, Artificial
  synthetic peptide
MYIEALDKYACK